(19) United States
(12) Patent Application Publication
(10) Pub. No.: US 2004/0010283 A1
(43) Pub. Date: Jan. 15, 2004

(54) Apparatus fot treatment of a nose bleed

(76) Inventor: Richard A. Buzard, Venetia, PA (US)

Correspondence Address: PAUL BOGDON, 220 GRANT STREET, PITTSBURGH, PA, 15219, US

(21) Appl. No.: 10/193,870

(22) Filed: Jul. 15, 2002

(51) Int. Cl. A61B 17/28

(52) U.S. Cl. 606/201; 606/205

(57) ABSTRACT

An apparatus for treatment of a nose bleed, in its preferred form having a pair of laterally spaced elongated arms pivotably connected to be selectively moved toward and away from each other like scissors or the like. A compression spring engages the arms to urge them into a desired position with respect to each other. Removable absorbent sponges are disposed on the upper ends of the arms and are arranged to engage both the septum of a nostril where typical nose bleeds would occur and the non-bleeding septum. The arms are squeezed to separate them and to insert the sponges into the nostrils and released to apply pressure on the bleeding and non-bleeding septums. The sponge engaging the bleeding area may be saturated with a vasoconstrictive agent to control the bleeding. That sponge may be removed and replaced with a sponge saturated with a local antiseptic and applied to formerly bleeding area in anticipation of cauterizing the same area using a sponge saturated with a cauterizing agent.

### BACKGROUND OF THE INVENTION

1. Field of Invention

This invention relates generally to an apparatus for treating and controlling nose bleeding.

2. Description of the Prior Art

Nose bleeds warranting medical attention have been controlled by methods and devices which have been ineffective. Expandable sponges or surgical gauze packs are typically inserted into a nostril to engage the bleeding area of the septum. Neither of those are able to apply sufficient pressure on the blood vessel to effectively control the bleeding. Various devices and apparatus have been proposed and described for treating nose bleeds. Kern and Westwood in U.S. Pat. No. 4,457,756 described a device for treating common nose bleeds usable by individuals lacking medical training. In that same U.S. Patent a number of different nose bleed treatment devices are described, most of which are somewhat complex in structure of the present invention. The prior devices, although appearing to provide useful treatment modalities for severe nose bleeds, have not appeared for use by medical personnel. The art has failed to provide a safe, simple, and reliable device or or apparatus which is simple in construction and use, inexpensive and effective in controlling nose bleeds.

The present invention provides a simple and effective apparatus for controlling nose bleeds, the device being sufficiently inexpensive to produce. It is configured as a permanent, non-disposable, or disposable structure useable with disposable sponges or the like for the various treatment steps typically taken in treating severe nose bleeds.

### SUMMARY OF THE INVENTION

The present invention provides an apparatus for treating and controlling any nose bleeds and particularly nose bleeds of a severe nature requiring medical attention. The preferred embodiment of the present apparatus provides a pair of laterally spaced elongated arms, pivotably connected to allow scissor-like movement toward and away from each other. Biasing means in the form of a compression spring engages the arms to urge them toward each other and to apply pressure on the bleeding vessel of a nasal septum. Removable absorbent sponges, or such like, are disposed on the upper end sections of the arms and typically one of the sponges would be saturated with a vasoconstrictive agent with the other being plain. The arms are squeezed by the user to separate the upper ends for insertion into the nostrils for engaging both the bleeding septum and non-bleeding septum. The arms are released resulting in pressure being applied to the bleeding, as well as nonbleeding areas. When the bleeding is controlled the apparatus may be removed from the nostrils, the bleed control sponge simply removed and replaced with a sponge saturated with a local anesthetic, if the vasoconstrictive agent is not combined with an anesthetic, and applied to the formerly bleeding area in anticipation of cauterizing the same area using a sponge saturated with a cauterizing agent.

The basic structure of the apparatus of the present invention is simple in construction, relatively inexpensive to produce and uncomplicated in use. The absorbent sponges are removable and easily replaced. The apparatus is modifiable in various ways, for example, the biasing means may be provided with an adjustment for selectively varying the tension force on the arms, and the absorbent sponge pivotably mounted on the arms for varying their orientation with respect to differently configured nostrils.

Various other advantages, details, and modifications of the present invention will become apparent on the following description of a certain present preferred embodiment proceeds.

### BRIEF DESCRIPTION OF THE DRAWINGS

In the accompanying drawings I show as certain present preferred embodiment of my invention in which:

FIG. 1 is a front elevation view of an apparatus for treating a nose bleed embodying this invention;

FIG. 2 is a side elevation view of this apparatus of the invention;

FIG. 3 is a view along the line III-III of FIG. 1;

FIG. 4 is a view along the line IV-IV of FIG. 1; and

FIG. 5 is a partial sectional view of the apparatus of FIG. 1 showing details of construction.

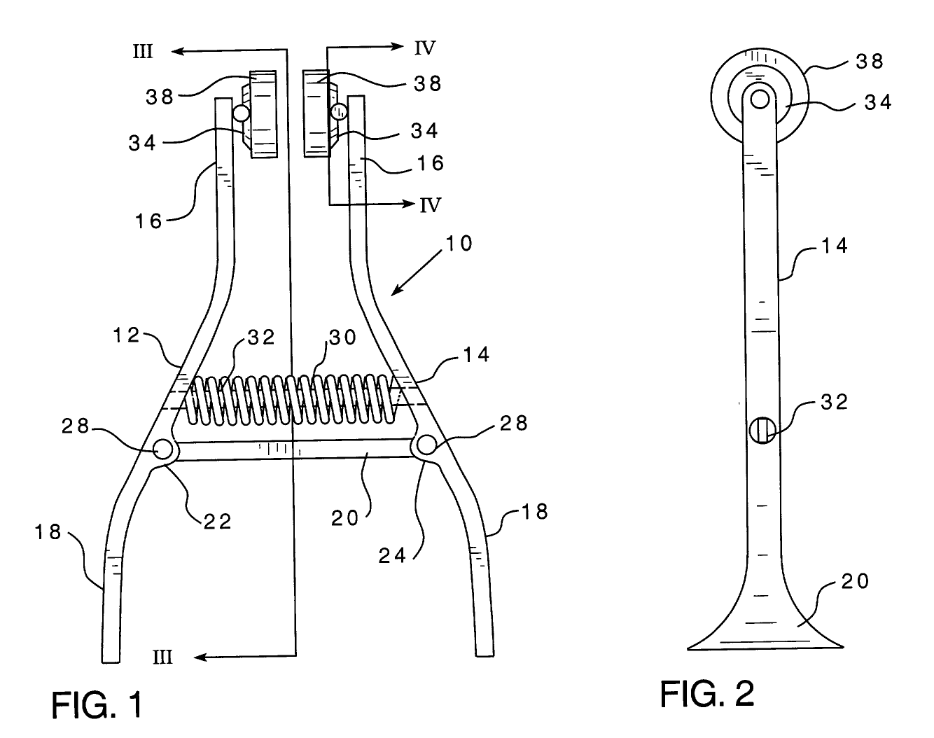

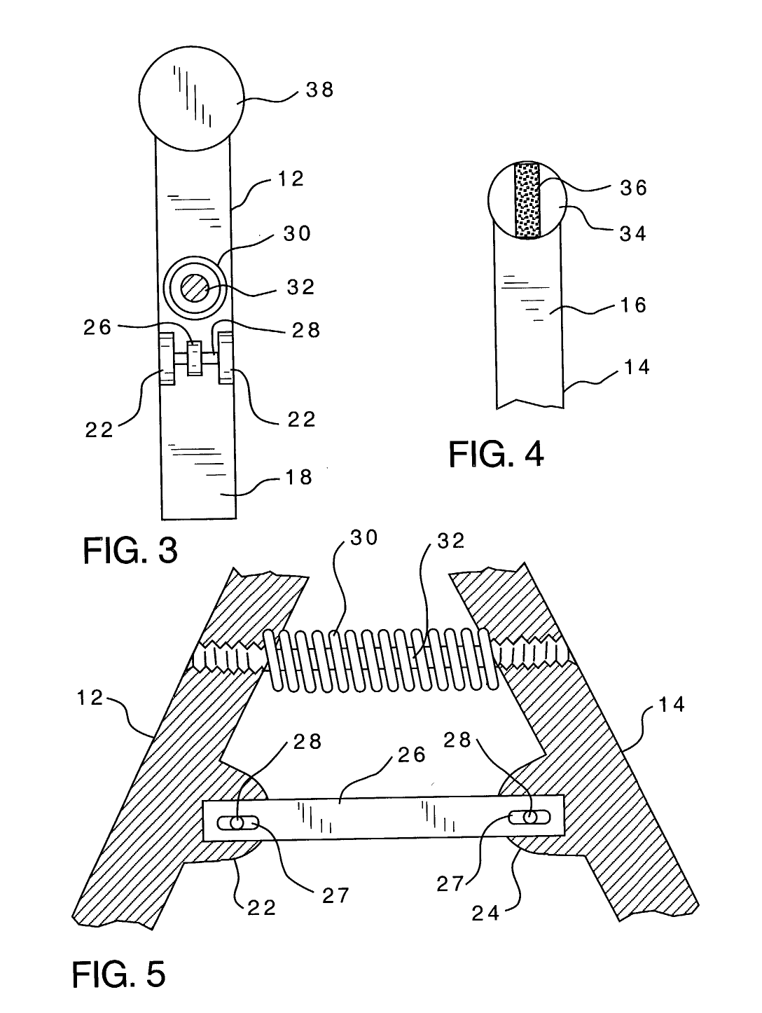

### DESCRIPTION OF A PREFERRED EMBODIMENT

Referring now to the drawings there is shown an apparatus 10 for treating a nose bleed embodying the present invention. The apparatus 10 includes a pair of identically shaped and sized elongated somewhat S-shaped arm members 12 and 14 each having an upper section 16 and lower section 18. Each lower section 18 has a flared end 20 with the remainder of each lower section sized to accommodate the hand of the user, the flared end 20 being in the nature of a stop for preventing the hand from slipping from the arm members 12 and 14. Each arm member 12 and 14 is provided with a clevis joint 22 and 24 on the interior thereof and at the upper end of the lower sections 18. An elongated, generally straight rod 26 is pivotably secured within slots 27 at each of its end sections to pivot pins 28 supported by the clevis joints 22 and 24, thereby connecting the arm members 12 and 14 for selective movement angularly toward and away from each other. FIG. 5 shows the connection arrangement of the rod 26 to the pivot pins 28 within the clevis joints 22 and 24 with the arrangement allowing limited lateral spacing of the arm members 12 and 14 with respect to each other.

A helical coil spring 30 is secured at its respective ends to intermediate sections of the interior of the arm member 12 and 14 and above the rod 26 and clevis joint 22 and 24 assemblies. The spring is in tension and urges force on the arm members 12 and 14 biasing them toward each other. As clearly shown in FIG. 5, an elongated adjustment bar 32 is disposed within the confines of the spring 30 with the respective ends threadedly received in the bodies of the arm members 12 and 14. The adjustment bar 32 may be selectively turned clockwise or counterclockwise to move the lateral positions of the arm members 12 and 14 with respect to each other thereby varying the tension force of the spring 30.

Arranged on the upper end sections 16 of the arm members 12 and 14 are identical mounting pads 34 each being pivotably connected to the upper end sections 16. The inner face of each mounting pad 34 is provided with an attachment strip 36 to which is removably attached an absorbent disc-shaped sponge 38. Each sponge 38 as removably secured to a mounting pad 34 is allowed limited rotation about an axis perpendicular to the longitudinal axis of an arm member 12 or 14.

The apparatus 10 of this invention is arranged and constructed such that the sponges 38 will fit into nostrils to engage the septums thereof. In its use for treating a nose bleed the user grasps the arm members 12 and 14 on the lower sections 18 thereof and squeezes against the bias of the spring 30 to spread the arm members 12 and 14 aligning the pivotable sponges 38 with the particular nostrils of a person experiencing a nose bleed. The sponge 38 engaging the bleed area could be saturated with a vasoconstrictive agent or combined vasoconstrictive and anesthetic agents, with the other sponge 38 being plain. The user would release the arm members 12 and 14 whereby the bleed and non-bleed septums would be under a desired pressure. After the bleeding is under control the user squeezes the arm members 12 and 14 and removes the attached sponges 38 from the nostrils. The sponge 38 used in controlling the bleeding may be removed and replaced with one saturated with a local anesthetic if not combined with a vasoconstrictive agent, the attached sponges 38 reinserted into the nostrils. The attached sponges 38 are removed and the formerly bleeding area may be cauterized by the insertion of attached sponges 38, one of which would be saturated with a cauterizing agent.

It would be clearly apparent to one skilled in the art of nose bleed treatment apparatus or devices that modifications could be made to the structure of this invention. The helical coil spring 30 could be replaced by a wound spring typically found on garden shears; sponges 38 could be cylindrically shaped and fitted over the upper end sections of the arm members 12 and 14. The arm members 12 and 14 could be oriented in a rotatable scissor-like arrangement. The apparatus 10 may be constructed as disposable or non-disposable. Various other modifications would be understood by those skilled in this art.

While I have shown and described a present preferred embodiment of this invention, it is to be distinctly understood that the invention is not limited thereto, but may be otherwise embodied within the scope of the following claims.

I claim:

1. Apparatus for treatment of a nose bleed, comprising:
a pair of laterally spaced elongated arm members having lower and upper end sections;
connector means intermediate said arm members for allowing movement of said arm members toward and away from each other;
biasing means engaging said arm members for urging said arm members toward each other;
absorbent means disposed on the upper end sections of at least one of said arm members; and
said arm members, connection means, and absorbent means constructed and arranged such that said absorbent means may be inserted into a nostril to contact a bleeding area under pressure sufficient to stop the flow of blood.

2. Apparatus for treatment of a nose bleed as set forth in claim 1 wherein said connector means pivotably engages said arm members for allowing movement of said arm members angularly toward and away from each other

3. Apparatus for treatment of a nose bleed as set forth in claim 1 wherein absorbent means are disposed on the upper end sections of each of said arm members.

4. Apparatus for treatment of a nose bleed as set forth in claim 1 wherein said absorbent means is selectively removable from said arm member.

5. Apparatus for treatment of a nose bleed as set forth in claim 1 including adjustment means engaging said biasing means for selectively varying the biasing force thereof.

6. Apparatus for treatment of a nose bleed as set forth in claim 1 including mounting means pivotably disposing said absorbent means on the upper section of said arm member for allowing rotation of said absorbent means about an axis perpendicular to the longitudinal axis of said arm member.
